(19) United States
(12) Patent Application Publication
(10) Pub. No.: US 2010/0249384 A1
(43) Pub. Date: Sep. 30, 2010

(54) IMMUNOGLOBULIN AGGREGATES

(76) Inventors: Stefan Hepbildikler, Muenchen (DE); Wolfgang Kuhne, Penzberg (DE); Eva Rosenberg, Muenchen (DE); Gerhard Winter, Penzberg (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 12/744,089

(22) PCT Filed: Nov. 27, 2008
(86) PCT No.: PCT/EP08/10060

§ 371 Date: May 21, 2010

(30) Foreign Application Priority Data
Nov. 29, 2007 (EP) 07023139.4

(51) Int. Cl. C07K 1/14

(52) U.S. Cl. 530/390.5

(57) ABSTRACT

The current invention reports a method for concentrating an immunoglobulin solution by tangential flow filtration wherein the immunoglobulin in polymeric form can be removed after the concentration.

The current invention is in the field of protein concentration, to be more precise it relates to the use of tangential flow filtration (TFF) for immunoglobulin concentration and immunoglobulin aggregate removal.

### BACKGROUND OF THE INVENTION

Proteins and especially immunoglobulins play an important role in today's medical portfolio. Expression systems for the production of recombinant polypeptides are well-known in the state of the art and are described by, e.g., Marino, M. H., Biopharm. 2 (1989) 18-33; Goeddel, D. V., et al., Methods Enzymol. 185 (1990) 3-7; Wurm, F., and Bernard, A., Curr. Opin. Biotechnol. 10 (1999) 156-159. Polypeptides for use in pharmaceutical applications are mainly produced in mammalian cells such as CHO cells, NS0 cells, Sp2/0 cells, COS cells, HEK cells, BHK cells, PER.C6® cells, and the like.

For human application every pharmaceutical substance has to meet distinct criteria. To ensure the safety of biopharmaceutical agents to humans, for example, nucleic acids, viruses, and host cell proteins, which would cause severe harm, have to be removed. To meet the regulatory specification one or more purification steps have to follow the manufacturing process. Among other, purity, throughput, and yield play an important role in determining an appropriate purification process.

Due to their chemical and physical properties, such as molecular weight and domain architecture, including secondary modifications, the downstream processing of immunoglobulins is very complicated. For example, concentrated solutions are required not only for formulated drugs but also for intermediates in downstream processing (DSP) to achieve low volumes for economic handling and application storage. Furthermore, fast concentration processes are favored to ensure smooth processes and short operating times. In this context imperfect tangential flow filtration (TFF) processes, especially after final purification steps, can cause sustained damage even affecting the final drug product. The correlation between shear stress and aggregation in tangential flow concentration processes for monoclonal antibody (mAb) intermediate solutions was investigated by Ahrer, K., et al., J. Membr. Sci. 274 (2006) 108-115. The influence on scalable process performance by selecting defined flow and pressure parameters was monitored (see e.g. Dosmar, M., et al., Bioprocess Int. 3 (2005) 40-50; Luo, R., et al., Bioprocess Int. 4 (2006) 44-46). Mahler, H.-C., et al., (Eur. J. Pharmaceut. Biopharmaceut. 59 (2005) 407-417) reported the induction and analysis of aggregates in a liquid IgG1-antibody formulation formed by different agitation stress methods. In U.S. Pat. No. 6,252,055 a concentrated monoclonal antibody preparation is reported. A method for producing a concentrated antibody preparation is reported in US 2006/0182740. A combined process including an ultrafiltration, a diafiltration, and a second ultrafiltration sequence is reported in US 2006/0051347. In EP 0 907 378 is reported a process for concentrating an antibody preparation using a cross-flow ultrafiltration with a fixed recirculation rate of 250 ml/min.

### SUMMARY OF THE INVENTION

One aspect of the current invention is a method for obtaining a concentrated immunoglobulin solution by tangential flow filtration, characterized in that said method comprises the following steps:
i) providing a solution containing an immunoglobulin to be concentrated wherein said immunoglobulin is present in said solution in monomeric and polymeric form, whereby the fraction of the polymeric soluble immunoglobulin form present in said provided solution has a first value of more than 2.5% when determined by size exclusion chromatography,
ii) concentrating said solution provided under i) by employing a tangential flow filtration,

iii) removing said polymeric immunoglobulin by filtration after the end of the tangential flow filtration and thereby obtaining a concentrated immunoglobulin solution,

whereby the fraction of said polymeric soluble form of said immunoglobulin is after step ii) larger than said first value and smaller than a second value which is 1.25 times the first value.

Another aspect of the current invention is a method for obtaining a concentrated immunoglobulin solution by tangential flow filtration, characterized in that said method comprises the following steps:
i) providing a solution containing an immunoglobulin to be concentrated wherein said immunoglobulin is present in said solution in monomeric and polymeric form, whereby the fraction of the polymeric soluble immunoglobulin form present in said provided solution is more than 2.5% when determined by size exclusion chromatography with a first number of particles with a size of more than 1 μm in said solution determined by light obscuration,
ii) concentrating said solution provided under i) by employing a tangential flow filtration with a constant Δp of 3.0 bar thereby obtaining a concentrated immunoglobulin solution with a second number of particles with a size of more than 1 μm in said solution determined by light obscuration,
   whereby said second number of particles with a size of more than 1 μm is less than 200 times said first number of particles with a size of more than 1 μm.

A further aspect of the current invention is a method for producing a heterologous immunoglobulin comprising the following steps:
a) providing a recombinant mammalian cell comprising one or more nucleic acids encoding a heterologous immunoglobulin,
b) cultivating said cell under conditions suitable for the expression of the heterologous immunoglobulin,
c) recovering the heterologous immunoglobulin from the recombinant mammalian cell or the culture medium,
d) concentrating the obtained aqueous, buffered solution comprising the heterologous immunoglobulin using a tangential flow filtration method with a constant Δp of 3.0 bar, whereby the number of particles in said concentrated solution with a size of more than 1 μm is smaller than 200 times the number of particles with a size of more than 1 μm in the solution prior to the concentrating when determining the number of particles by light obscuration.

Still a further aspect of the current invention is a method for removing immunoglobulin aggregates from an immunoglobulin solution, characterized in that said method comprises the following steps:
i) providing a solution containing said immunoglobulin to be concentrated, wherein said immunoglobulin is present in said solution in monomeric and polymeric form, or
providing a solution containing an immunoglobulin to be concentrated, wherein in said solution the formation of said immunoglobulin in polymeric form is induced by the application of heat,
ii) concentrating said solution provided under i) by employing a tangential flow filtration with a constant Δp of 3.0 bar, and a constant transmembrane pressure of 0.6 bar,
iii) removing immunoglobulin aggregates from the immunoglobulin solution obtained in step ii) by filtration with a 0.2 μm pore size filter,
   whereby the number of particles in said concentrated solution of step ii) with a size of more than 1 μm is smaller than 200 times the number of particles with a size of more than 1 μm in the solution prior to the concentrating when determining the number of particles by light obscuration.

A final aspect of the current invention is a method for the reduction of the formation of immunoglobulin in polymeric soluble form during a concentration step with tangential flow filtration, wherein the reduction is achieved by the addition, supplementation, or generation of immunoglobulin in polymeric soluble form prior to the start of the concentration step.

In one embodiment of the method according to the invention the number of particles in said concentrated solution of with a size of more than 5 μm is smaller than 100 times the number of particles with a size of more than 5 μm in the solution prior to the concentrating when determining the number of particles by light obscuration. In a further embodiment the fraction of said polymeric soluble immunoglobulin form is more than 5% when determined as the area under the curve of the peaks eluted prior to the peak of the monomeric immunoglobulin in a size exclusion chromatogram of said solution. In another embodiment comprises the method according to the invention prior to or after step d) the following step:
e) purifying the aqueous, buffered solution containing the heterologous immunoglobulin.

In one embodiment the heterologous immunoglobulin is a complete immunoglobulin, or an immunoglobulin fragment, or an immunoglobulin conjugate. In a further embodiment the mammalian cell is a CHO cell, a BHK cell, or a PER.C6® cell. In another embodiment the determining by light obscuration is at a protein concentration of 90 mg/ml. In one embodiment the fraction of said polymeric soluble form of said immunoglobulin is larger than said first value and smaller than a second value which is 1.10 times the first value. In one embodiment one aspect of the invention is a method for obtaining a concentrated immunoglobulin solution free of insoluble immunoglobulin aggregates by tangential flow filtration and said concentrated immunoglobulin solution is free of insoluble immunoglobulin aggregates. In another embodiment said method is for obtaining a concentrated immunoglobulin solution free of insoluble immunoglobulin aggregates and soluble immunoglobulin aggregates of more than 5 μm by tangential flow filtration, in which said concentrated immunoglobulin solution is free of insoluble immunoglobulin aggregates and free of soluble immunoglobulin aggregates of more than 5 μm and said removing said polymeric immunoglobulin is by filtration with a filter with 1.0 μm pore size of less, in one embodiment of 0.2 μm pore size. In a further embodiment the concentration of the immunoglobulin after the concentrating is more than 80 mg/ml, in another embodiment more than 90 mg/ml, in still another embodiment more than 100 mg/ml. In one embodiment the concentration of the immunoglobulin after the concentrating is less than 275 mg/ml, or less than 180 mg/ml, or less than 130 mg/ml. In another embodiment said filtrating the concentrated immunoglobulin solution is with a filter with a pore size of 1 μm or less in order to remove the immunoglobulin in soluble aggregated form of more than 5 μm and to remove the immunoglobulin in insoluble aggregated form. In one embodiment said polymeric immunoglobulin form is soluble aggregated and insoluble aggregated immunoglobulin. In one embodiment said immunoglobulin in polymeric form is a soluble aggregated immunoglobulin form. In one embodiment the fraction of the polymeric soluble immunoglobulin form is less than 25%, in another embodiment less than 15%, and in still another embodiment less than 10%.

### DETAILED DESCRIPTION OF THE INVENTION

The current invention provides a method for obtaining a concentrated immunoglobulin solution free of immunoglobulin aggregates by tangential flow filtration comprising the following steps:
i) providing a solution containing an immunoglobulin to be concentrated wherein said immunoglobulin is present in said solution in monomeric and polymeric form, whereby the fraction of said soluble polymeric form is more than 2.5% when determined as the area under the curve of the peaks eluted prior to the peak of the monomeric immunoglobulin in a size exclusion chromatogram of said provided solution, i.e. of the soluble high molecular weight (HMW) forms,
ii) concentrating said solution provided under i) by employing a tangential flow filtration, whereby during the tangential flow filtration the number of immunoglobulin molecules contained in the immunoglobulin in polymeric form increases, preferably until insoluble particles are formed,
iii) removing said insoluble, immunoglobulin in polymeric form by a filtration step after the end of the tangential flow filtration and thereby obtaining a concentrated immunoglobulin solution free of immunoglobulin aggregates.

An anti-IL-1R antibody (see WO 2005/023872) was available in sufficient quantities in our laboratories at the time of the invention and, therefore, the current invention is exemplified with this immunoglobulin. The invention is likewise in general practicable with other immunoglobulins. This exemplified description is done only by way of example and not by way of limitation of the invention. These examples are provided to aid the understanding of the present invention, the true scope of which is set forth in the appended claims.

The terms “tangential flow filtration” or “TFF”, which are used interchangeably within the current invention, denote a filtration process wherein a solution containing a polypeptide to be concentrated flows along, i.e. tangential, to the surface of a filtration membrane. The filtration membrane has a pore size with a certain cut off value. In one embodiment the cut off value is in the range of from 20 kDa to 50 kDa, in another embodiment of 30 kDa. This filtration process is a kind of an ultrafiltration process. The term “cross-flow” denotes the flow of the solution to be concentrated tangential to the membrane (retentate flow).

The terms “transmembrane pressure” or “TMP”, which are used interchangeably within the current invention, denote the pressure which is applied to drive the solvent and components smaller than the cut-off value of the filtration membrane through the pores of the filtration membrane. In one embodiment the transmembrane pressure in the methods according to the current invention is 0.6 bar. The transmembrane pressure is an average pressure of the inlet, outlet and permeate and can be calculated as:

$$TMP = \frac{( {p}_{in} + {p}_{out} )}{2} - {p}_{permeate}$$

The term “immunoglobulin” refers to a protein consisting of one or more polypeptide(s) substantially encoded by immunoglobulin genes. The recognized immunoglobulin genes include the different constant region genes as well as the myriad immunoglobulin variable region genes. Immunoglobulins may exist in a variety of formats, including, for example, Fv, Fab, and F(ab)₂ as well as single chains (scFv) or diabodies (e.g. Huston, J. S., et al., Proc. Natl. Acad. Sci. USA 85 (1988) 5879-5883; Bird, R. E., et al., Science 242 (1988) 423-426; in general, Hood, L. E., et al., Immunology, The Benjamin N.Y., 2nd edition (1984); and Hunkapiller, T., and Hood, L., Nature 323 (1986) 15-16).

The term “complete immunoglobulin” denotes an immunoglobulin which comprises two so called light immunoglobulin chain polypeptides (light chains) and two so called heavy immunoglobulin chain polypeptides (heavy chains). Each of the heavy and light immunoglobulin chain polypeptides of a complete immunoglobulin contains a variable domain (variable region) (generally the amino terminal portion of the polypeptide chain) comprising binding regions that are able to interact with an antigen. Each of the heavy and light immunoglobulin chain polypeptides of a complete immunoglobulin comprises a constant region (generally the carboxyl terminal portion). The constant region of the heavy chain mediates the binding of the antibody i) to cells bearing a Fc gamma receptor (FcγR), such as phagocytic cells, or ii) to cells bearing the neonatal Fc receptor (FcRn) also known as Brambell receptor. It also mediates the binding to some factors including factors of the classical complement system such as component (C1q). The variable domain of an immunoglobulin's light or heavy chain in turn comprises different segments, i.e. four framework regions (FR) and three hypervariable regions (CDR).

The term “immunoglobulin fragment” denotes a polypeptide comprising at least one domain selected from the variable domain, the C_{H}1 domain, the hinge-region, the C_{H}2 domain, the C_{H}3 domain, or the C_{H}4 domain of a heavy chain, or the variable domain or the C_{L} domain of a light chain. Also enclosed are derivatives and variants thereof. For example, a variable domain, in which one or more amino acids or amino acid regions are deleted, may be present.

The term “immunoglobulin conjugate” denotes a polypeptide comprising at least one domain of an immunoglobulin heavy or light chain conjugated via a peptide bond to a further polypeptide. The further polypeptide is a non-immunoglobulin peptide, such as a hormone, or growth receptor, or antifusogenic peptide, or complement factor, or the like. In one embodiment said immunoglobulin conjugate contains an immunoglobulin molecule covalently linked to two or four non-immunoglobulin polypeptides.

General chromatographic methods and their use are known to a person skilled in the art. See for example, Chromatography, 5^{th} edition, Heftmann, E. (ed.), Part A: Fundamentals and Techniques, Elsevier Science Publishing Company, New York, (1992); Deyl, Z. (ed.), Advanced Chromatographic and Electromigration Methods in Biosciences, Elsevier Science BV, Amsterdam, The Netherlands, (1998); Poole, C. F., and Poole, S. K., Chromatography Today, Elsevier Science Publishing Company, New York, (1991); Scopes, R. K., Protein Purification: Principles and Practice, Springer Verlag, New York, (1982); Sambrook, J., et al., (ed.), Molecular Cloning: A Laboratory Manual, second edition, Cold Spring Harbor Laboratory Press, Cold Spring Harbor, N.Y., (1989); or Current Protocols in Molecular Biology, Ausubel, F. M., et al., (eds), John Wiley & Sons, Inc., New York.

For the purification of recombinantly produced heterologous immunoglobulins often a combination of different column chromatography steps is employed. Generally a protein A affinity chromatography is followed by one or two additional separation steps. The final purification step is a so called “polishing step” for the removal of trace impurities and contaminants like aggregated immunoglobulins, residual HCP (host cell protein), DNA (host cell nucleic acid), viruses, or endotoxins. For this polishing step often an anion exchange material in a flow-through mode is used.

Different methods are well established and widespread used for protein recovery and purification, such as affinity chromatography with microbial proteins (e.g. protein A or protein G affinity chromatography), ion exchange chromatography (e.g. cation exchange (carboxymethyl resins), anion exchange (amino ethyl resins) and mixed-mode exchange), thiophilic adsorption (e.g. with beta-mercaptoethanol and other SH ligands), hydrophobic interaction or aromatic adsorption chromatography (e.g. with phenyl-sepharose, aza-arenophilic resins, or m-aminophenylboronic acid), metal chelate affinity chromatography (e.g. with Ni(II)- and Cu(II)-affinity material), size exclusion chromatography, and electrophoretical methods (such as gel electrophoresis, capillary electrophoresis) (Vijayalakshmi, M. A., Appl. Biochem. Biotech. 75 (1998) 93-102).

The term “heterologous immunoglobulin” denotes an immunoglobulin which is not naturally produced by a mammalian cell. The immunoglobulin produced according to the method of the invention is produced by recombinant means. Such methods are widely known in the state of the art and comprise protein expression in eukaryotic cells with subsequent recovery and isolation of the heterologous immunoglobulin, and usually purification to a pharmaceutically acceptable purity. For the production, i.e. expression, of an immunoglobulin a nucleic acid encoding the light chain and a nucleic acid encoding the heavy chain are inserted each into an expression cassette by standard methods. Nucleic acids encoding immunoglobulin light and heavy chains are readily isolated and sequenced using conventional procedures. Hybridoma cells can serve as a source of such nucleic acids. The expression cassettes may be inserted into an expression plasmid(s), which is (are) then transfected into a cell, which does not otherwise produce immunoglobulins. Expression is performed in appropriate prokaryotic or eukaryotic cells and the immunoglobulin is recovered from the cells after lysis or from the culture supernatant.

The term “solution containing an immunoglobulin to be concentrated” as used within the current application denotes an aqueous, buffered solution containing a complete immunoglobulin, an immunoglobulin fragment, or an immunoglobulin conjugate. This solution may be, e.g., a culture supernatant, or a column chromatography eluate, or a polished immunoglobulin solution.

“Heterologous DNA” or “heterologous polypeptide” refers to a DNA molecule or a polypeptide, or a population of DNA molecules or a population of polypeptides, that do not exist naturally within a given host cell. DNA molecules heterologous to a particular host cell may contain DNA derived from the host cell species (i.e. endogenous DNA) so long as that host DNA is combined with non-host DNA (i.e. exogenous DNA). For example, a DNA molecule containing a non-host DNA segment encoding a polypeptide operably linked to a host DNA segment comprising a promoter is considered to be a heterologous DNA molecule. Conversely, a heterologous DNA molecule can comprise an endogenous structural gene operably linked with an exogenous promoter.

A peptide or polypeptide encoded by a non-host DNA molecule is a “heterologous” peptide or polypeptide.

The term “under conditions suitable for the expression of the heterologous immunoglobulin” denotes conditions which are used for the cultivation of a mammalian cell expressing an immunoglobulin and which are known to or can easily be determined by a person skilled in the art. It is also known to a person skilled in the art that these conditions may vary depending on the type of mammalian cell cultivated and type of immunoglobulin expressed. In general the mammalian cell is cultivated at a temperature of from 20° C. to 40° C., and for a period of time sufficient to allow effective protein production of the immunoglobulin, e.g. of from 4 to 28 days.

The current invention provides a method for obtaining a concentrated immunoglobulin solution substantially free of immunoglobulin aggregates by tangential flow filtration comprising the following steps:
i) providing a solution containing an immunoglobulin to be concentrated wherein said immunoglobulin is present in said solution in monomeric and polymeric form, whereby the fraction of said soluble polymeric form is more than 2.5% when determined as the area under the curve of the peaks eluted prior to the peak of the monomeric immunoglobulin in a size exclusion chromatogram of said solution,
ii) concentrating said solution provided under i) by employing a tangential flow filtration, whereby said soluble polymeric form of said immunoglobulin does not increase by more than 25% when determined as the area under the curve of the peaks eluted prior to the peak of the monomeric immunoglobulin in a size exclusion chromatogram of said concentrated solution,
iii) removing polymeric insoluble immunoglobulin by filtration after the end of the tangential flow filtration and thereby obtaining a concentrated immunoglobulin solution substantially free of immunoglobulin aggregates.

In other words, the current invention comprises a method for obtaining a concentrated immunoglobulin solution by tangential flow filtration, characterized in that said method comprises the following steps:
i) providing a solution containing an immunoglobulin to be concentrated wherein said immunoglobulin is present in said solution in monomeric and polymeric form, whereby the fraction of the polymeric soluble immunoglobulin form present in said provided solution has a first value of more than 2.5% when determined by size exclusion chromatography,
ii) concentrating said solution provided under i) by employing a tangential flow filtration,
iii) removing said polymeric immunoglobulin by filtration after the end of the tangential flow filtration and thereby obtaining a concentrated immunoglobulin solution,
   whereby the fraction of said polymeric soluble form of said immunoglobulin is after step ii) larger than said first value and smaller than a second value which is 1.25 times the first value.

The term “substantially free” denotes that a preparation of an immunoglobulin contains at least 50% (w/w) of the immunoglobulin in monomeric form, in one embodiment at least 75% of the immunoglobulin in monomeric form, in another embodiment at least 90% of the immunoglobulin in monomeric form, or in a further embodiment more than 95% of the immunoglobulin in monomeric form.

The term “does not increase by more than 10%” as used within this application denotes that the fraction of the immunoglobulin in polymeric soluble form does not increase by more than 10%. For example if the fraction of the immunoglobulin in polymeric soluble form is 7.5% prior to the tangential flow filtration determined by size exclusion chromatography this term denotes that the fraction does not increase by more than 0.75%, that is to 8.25% at maximum.

The term “immunoglobulin in monomeric form” as used within this application denotes immunoglobulin molecules which are not associated either covalently or non-covalently with one or more other immunoglobulin molecules. This does not exclude that the immunoglobulin molecule is associated either covalently or non-covalently with one or more not-immunoglobulin molecules, such as carbohydrates, chromatin etc.

The terms “immunoglobulin in polymeric form” and “immunoglobulin in aggregated form” as used within this application denotes immunoglobulin molecules which are associated either covalently or non-covalently with one or more immunoglobulin molecules. These associated immunoglobulin molecules may be of the same immunoglobulin molecule or different immunoglobulin molecules. This does not exclude that the immunoglobulin molecule is associated either covalently or non-covalently with one or more not-immunoglobulin molecules, such as carbohydrates, chromatin etc. The term “polymeric soluble form” or “high molecular weight (HMW) form”, which can be used interchangeably within this application, denote polymeric, i.e. aggregated, immunoglobulin, whereby said aggregate is still soluble in an aqueous buffered solution. The term “polymeric insoluble form” denotes polymeric, i.e. aggregated, immunoglobulin whereby said aggregate is not soluble in an aqueous buffered solution.

In step i) of the method according to the invention is the immunoglobulin present in monomeric form and in polymeric form, whereby these two forms are soluble in the solution. In step ii) of the method is the provided immunoglobulin solution containing the immunoglobulin in monomeric and polymeric form concentrated using a tangential flow filtration method. It has now surprisingly been found that if a solution contains an immunoglobulin in polymeric but soluble form prior to a concentration step with tangential flow filtration the number of immunoglobulin molecules, i.e. the size of the particle, contained in the immunoglobulin in polymeric form increases until the immunoglobulin in polymeric form is no longer soluble in the solution. Concomitantly almost no new soluble immunoglobulin in polymeric form is formed during the tangential flow filtration. Thus, it has been found that in immunoglobulin solutions containing immunoglobulin in polymeric soluble form prior to a concentration step with tangential flow filtration the contained immunoglobulin in polymeric soluble form further agglomerates additional immunoglobulin molecules from the solution resulting in a reduced formation of new immunoglobulin in polymeric soluble form. Thus, this results in immunoglobulin in polymeric insoluble form, which can be removed from the concentrated immunoglobulin solution after the concentration step by a simple filtration step.

Another aspect of the current invention is a method for obtaining a concentrated immunoglobulin solution by tangential flow filtration comprising the following steps:
i) providing a solution containing an immunoglobulin to be concentrated wherein said immunoglobulin is present in said solution in monomeric and polymeric form,
ii) concentrating said solution provided under i) by employing a tangential flow filtration with a Δp of 3.0 bar,
   whereby the number of particles with a size of more than 1 μm in said solution does not increase during the concentrating step by more than a factor of 200 when the number of particles is determined by light obscuration. In one embodiment the number of particles is determined by light obscuration at a protein concentration of 90 mg/ml. In one embodiment the number of particles with a size of more than 5 μm does not increase by more than a factor of 100 when the number of particles is determined by light obscuration. In another embodiment is the fraction of said polymeric form of said immunoglobulin more than 5% when determined as the area under the curve of the peaks eluted prior to the peak of the monomeric immunoglobulin in a size exclusion chromatogram of said solution. In a further embodiment is the concentration of the immunoglobulin after the concentrating more than 80 mg/ml, in one embodiment 90 mg/ml or more. In another embodiment the transmembrane pressure during the tangential flow filtration is constant at 0.6 bar.

Another aspect of the invention is a method for producing a heterologous immunoglobulin comprising the following steps:
a) providing a recombinant mammalian cell comprising one or more nucleic acids encoding a heterologous immunoglobulin,
b) cultivating said cell under conditions suitable for the expression of the heterologous immunoglobulin,
c) recovering the heterologous immunoglobulin from the recombinant mammalian cell or the culture medium,
d) concentrating the recovered aqueous, buffered solution comprising the heterologous immunoglobulin using a tangential flow filtration method with a constant Δp of 3.0 bar, whereby the number of particles in said solution with a size of more than 1 μm does not increase by more than a factor of 200 when the number of particles is determined by light obscuration.

In one embodiment the number of particles is determined at a protein concentration of 90 mg/ml. In a further embodiment the heterologous immunoglobulin is a complete immunoglobulin, or an immunoglobulin fragment, or an immunoglobulin conjugate. In still another embodiment the mammalian cell is a CHO cell, a BHK cell, or a PER.C6® cell.

The term “recombinant mammalian cell” refers to a cell into which a nucleic acid, e.g. encoding a heterologous polypeptide, can be or is introduced/transfected. The term “cell” includes cells which are used for the expression of nucleic acids. In one embodiment the mammalian cell is a CHO cell (e.g. CHO K1, CHO DG44), or a BHK cell, or a NS0 cell, or a SP2/0 cell, or a HEK 293 cell, or a HEK 293 EBNA cell, or a PER.C6® cell, or a COS cells. In another embodiment the mammalian cell is a CHO cell, or a BHK cell, or a PER.C6® cell. As used herein, the expression “cell” includes the subject cell and its progeny. Thus, the term “recombinant cell” includes the primary transfected cell and cultures including the progeny cells derived there from without regard to the number of transfers. It is also understood that all progeny may not be precisely identical in DNA content, due to deliberate or inadvertent mutations. Variant progeny that have the same function or biological activity as the originally transformed cell are included.

The term “buffered” as used within this application denotes a solution in which changes of pH due to the addition or release of acidic or basic substances is leveled by a buffer substance. Any buffer substance resulting in such an effect can be used. In one embodiment pharmaceutically acceptable buffer substances are used, such as e.g. phosphoric acid or salts thereof, acetic acid or salts thereof, citric acid or salts thereof, morpholine or salts thereof, 2-(N-morpholino) ethanesulfonic acid or salts thereof, histidine or salts thereof, glycine or salts thereof, arginine or salts thereof, or TRIS (hydroxymethyl aminomethane) or salts thereof. In one embodiment the buffer substance is phosphoric acid or salts thereof, acetic acid or salts thereof, or citric acid or salts thereof, or histidine or salts thereof, or arginine or salts thereof. Optionally the buffered solution may comprise an additional salt, such as e.g. sodium chloride, and/or sodium sulphate, and/or potassium chloride, and/or potassium sulfate, and/or sodium citrate, and/or potassium citrate. In one embodiment of the invention the pH value of the buffered aqueous solution is of from pH 3.0 to pH 10.0, in another embodiment of from pH 3.0 to pH 7.0, in a further embodiment of from pH 4.0 to pH 6.0, and in still another embodiment of from pH 4.5 to pH 5.5.

In another embodiment comprises the method prior to, i.e. before, or after step d) the following step:
e) purifying the aqueous, buffered solution containing the heterologous immunoglobulin.

The purification in step e) can be by different methods and techniques, such as a chromatography step, or a combination of different or similar chromatography steps, or precipitation, or salting out, or ultrafiltration, or diafiltration, or lyophilization, or buffer change, or combinations thereof, or the like.

In another embodiment the heterologous immunoglobulin is a complete immunoglobulin, or an immunoglobulin fragment, or an immunoglobulin conjugate. In one embodiment the mammalian cell is a CHO cell, a BHK cell, a NS0 cell, a Sp2/0 cell, a COS cell, a HEK cell, or a PER.C6® cell.

Still another aspect of the current invention is a method for obtaining a concentrated immunoglobulin solution, comprising the following steps:
i) providing a solution containing an immunoglobulin to be concentrated wherein said immunoglobulin is present in said solution in monomeric and polymeric form,
ii) concentrating said solution by employing a tangential flow filtration, and producing thereby a concentrated immunoglobulin solution,
iii) filtrating the concentrated immunoglobulin solution obtained under ii) in order to remove the immunoglobulin in polymeric form.

Thus, it has been found that during the tangential flow filtration of immunoglobulin solutions containing said immunoglobulin in monomeric form and in polymeric form the formation of additional soluble immunoglobulin in polymeric form is reduced whereas the already contained immunoglobulin in polymeric form which is soluble in the solution is transferred to an immunoglobulin in polymeric form which is insoluble in the solution. Thus, the number of immunoglobulin molecules associated with each other in the immunoglobulin in polymeric form increases during the concentration of the immunoglobulin solution with tangential flow filtration until the immunoglobulin in polymeric form is no longer soluble in the solution. Thus, another aspect of the current invention is a method for the reduction of the formation of soluble immunoglobulin in polymeric form during a concentration step with tangential flow filtration, wherein the reduction is achieved by the addition, supplementation, or generation of soluble immunoglobulin in polymeric form prior to the start of the tangential flow filtration. The immunoglobulin in polymeric form may be, e.g., generated by heat stress.

The following examples and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 Turbidity during concentration via different concentration modes; X-axis: concentration in mg/ml, Y-axis: turbidity determined at 350 nm.

FIG. 2 Number of particles>1 μm per ml solution during concentration via different concentration modes; X-axis: concentration factor (CF), Y-axis: 10⁶ particles/ml>1 μm.

FIG. 3 Stained insoluble aggregates out of concentrated anti-IL-1R antibody solutions (left: Δp 3.0 bar; right: Δp 1.2 bar).

FIG. 4 Increase in high molecular weight forms (HMWs) with respect to the HMWs present in the sample before concentration; X-axis: 1: with low HMW content (approximately 0.6%) prior to the TFF with Δp 1.2 bar; 2: with high HMW content (approximately 7.2%) prior to the TFF with Δp 1.2 bar; 3: with low HMW content prior to the TFF with Δp 3.0 bar; 4: with high HMW content prior to the TFF with Δp 3.0 bar; Y-axis increase in HMWs in %.

FIG. 5 Number of particles per ml in the solutions before concentration and after concentration with a Δp of 1.2 bar or 3.0 bar, respectively; X-axis: 1: before concentration with low HMW content; 2: before concentration with high HMW content; 3: after concentration of solution with low HMW content with Δp of 1.2 bar; 4: after concentration of solution with high HMW content with Δp of 1.2 bar; 5: after concentration of solution with low HMW content with Δp of 3.0 bar; 6: after concentration of solution with high HMW content with Δp of 3.0 bar; left Y-axis: 10⁵ particles per ml; right Y-axis: particles per ml for size >25 μm.

FIG. 6 For material with a high initial high molecular weight form content a species at about 5000 nm is observable beside decreased intensity for the monomer (before concentration only one signal); X-axis: particle size in nm; Y-axis: relative intensity in %; squares: concentrate of a low HMW content solution at Δp of 1.2 bar; diamond: concentrate of a high HMW content solution at Δp of 1.2 bar; triangle: concentrate of a low HMW content solution at Δp of 3.0 bar; circle: concentrate of a high HMW content solution at Δp of 3.0 bar.

### Example 1

### Methods

### a) Turbidity Measurement.

The photometric absorbance was determined at 350 nm and 550 nm, where no intrinsic chromophores in the antibody solution absorb (UV-VIS spectrophotometer Evolution 500, Thermo Fisher Scientific, Waltham, USA). The samples were measured undiluted. As a reference medium the appropriate buffer solution was used. Every measurement was conducted three times.

### b) Size-Exclusion-HPLC.

The chromatography was conducted with a Tosoh Haas TSK 3000 SWXL column on a Summit HPLC system (Dionex, Idstein, Germany). The elution peaks were monitored at 280 nm by a UV diode array detector (Dionex). After dissolution of the concentrated samples to 1 mg/ml the column was washed with a buffer consisting of 200 mM potassium dihydrogen phosphate and 250 mM potassium chloride pH 7.0 until a stable baseline was achieved. The analyzing runs were performed under isocratic conditions using a flow rate of 0.5 ml/min. over 30 minutes at room temperature. The chromatograms were integrated manually with Chromeleon (Dionex, Idstein, Germany). Aggregation in % was determined by comparing the area under the curve (AUC) of high molecular weight forms with the AUC of the monomer peak.

### c) Light Obscuration.

To monitor the particle burden in a range of 1-200 μm a SVSS-C particle analyzer was used (PAMAS Partikelmess- and Analysesysteme, Rutesheim, Germany). The system was calibrated according to the requirements of US Pharmacopeia Vol. 24, <788>, with near-monosize polystyrene spheres. Three measurements of a volume of 0.5 ml with a pre-flushing volume of 0.5 ml were performed. Results were calculated as mean value and referred to a sample volume of 1.0 ml. The number of particles counted was within the sensor's concentration limit.

### d) Dynamic Light Scattering (DLS)

DLS is a non-invasive technique for measuring particle size, typically in the sub-micron size range. In the current invention the Zetasizer Nano S apparatus (Malvern Instruments, Worcestershire, UK) with a temperature controlled quartz cuvette (25° C.) was used for monitoring a size range between 1 nm and 6 μm. The intensity of the back scattered laser light was detected at an angle of 173°. The intensity fluctuates at a rate that is dependent upon the particle diffusion speed, which in turn is governed by particle size. Particle size data can therefore be generated from an analysis of the fluctuation in scattered light intensity (Dahneke, B. E. (ed), Measurement of Suspended Particles by Quasielectric Light Scattering, Wiley Inc. (1983); Pecora, R., Dynamic Light Scattering: Application of Photon Correlation Spectroscopy, Plenum Press (1985)). The size distribution by intensity was calculated using the multiple narrow mode of the DTS software (Malvern). Experiments were conducted with undiluted samples.

### e) Staining Method for Detection of Insoluble Aggregates

The concentrated antibody solution was filtered through a 0.22 μm cellulose acetate filter membrane (Sartorius, Göttingen, Germany) and the retained particles were stained with Reversible Protein Detection Kit solution from Sigma-Aldrich (Steinheim, Germany). The membrane was examined after washing with buffer under a stereomicroscope MZ 12 (Leica, Wetzlar, Germany) equipped with a digital camera DC 100 (Leica) under 80-times magnification (see e.g. Li, B., et al., J. Pharmaceutical Sci. 96 (2007) 1840-1843).

### Example 2

### Tangential Flow Filtration

A conditioned and filtered histidine-buffered aqueous solution (pH 5.8) of an anti-IL-IR antibody was concentrated twenty fold up to 100 mg/ml by use of an automated TFF system ÄKTAcrossflow™ (GE Healthcare, Amersham Bioscience AB, Uppsala, Sweden) by employing a scalable flat sheet cassette (Sartorius, Göttingen, Germany) with a Hydrosart™ membrane of regenerated cellulose, with a nominal molecular weight cut-off of 30 kDa, a membrane area of 0.02 m² and a total membrane loading of about 400 g/m².

The target concentration was set to 90 mg/ml. Different Δp parameters were tested and were the following:

Method 1: transmembrane pressure=0.6 bar
cross-flow=90 ml/min
Δp=1.2 bar
   Method 2: transmembrane pressure=0.6 bar
Δp=3.0 bar.

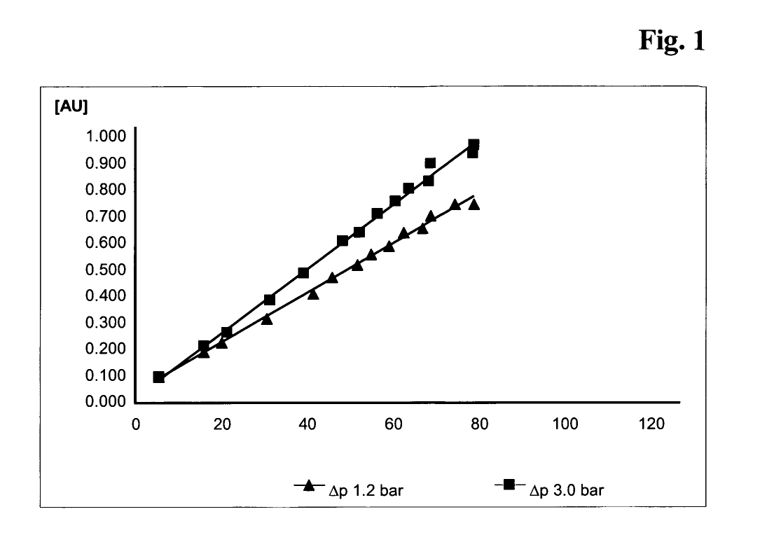

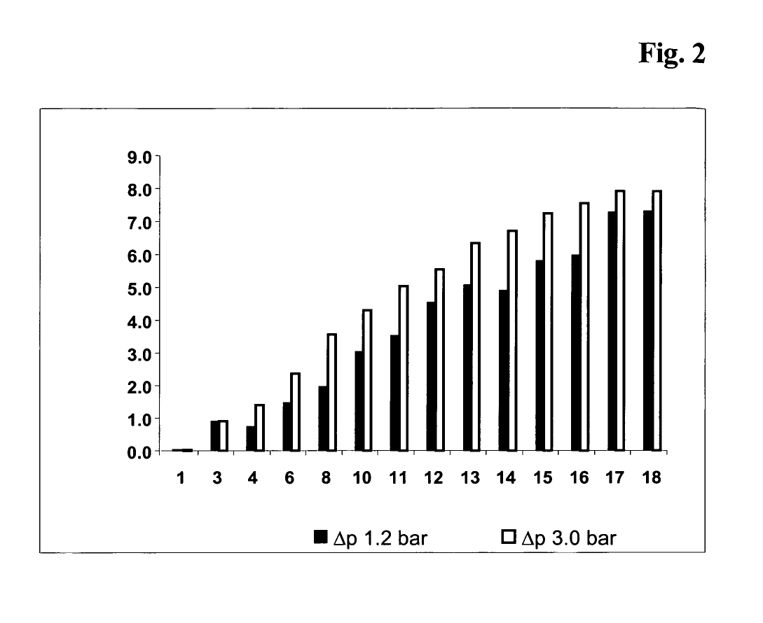

Turbidity measurements (FIG. 1), LO results (FIG. 2, Table 1) in the course of concentration process showed that enhanced formation of immunoglobulin in aggregated, insoluble form was depending on the applied shear stress.

TABLE 1
Light Obscuration-Analysis data of number of particles depending on the
Δp value and the number of aggregated forms in the beginning of the TFF

| | particle size | >1 μm | >5 μm | >10 μm | >25 μm |
|---|---|---|---|---|---|
| number of particles/ | before concentration, solution | 8110 | 271 | 71 | 3 |
| ml solution | containing a low number of aggregated | | | | |
| | forms (insoluble) | | | | |
| | before concentration, solution | 15499 | 472 | 93 | 5 |
| | containing a high number of aggregated | | | | |
| | forms (insoluble) | | | | |
| | TFF with Δp = 1.2 bar of low number | 11528874 | 261475 | 25335 | 0 |
| | aggregate solution | | | | |
| | TFF with Δp = 1.2 bar of high number | 29284408 | 381841 | 41169 | 667 |
| | aggregate solution | | | | |
| | before concentration, solution | 15845 | 213 | 54 | 7 |
| | containing a low number of aggregated | | | | |
| | forms | | | | |
| | before concentration, solution | 332595 | 7661 | 1091 | 66 |
| | containing a high number of aggregated | | | | |
| | forms | | | | |
| | TFF with Δp = 3.0 bar of low number | 27611698 | 207838 | 21835 | 333 |
| | aggregate solution | | | | |
| | TFF with Δp = 3.0 bar of high number | 46218164 | 707471 | 99469 | 1867 |
| | aggregate solution | | | | |

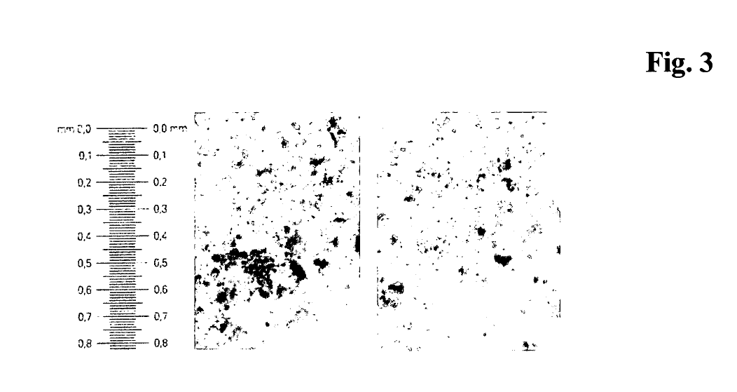

The visual easily detectable burden of insoluble immunoglobulin in polymeric form in concentrated solutions by using a filtration-staining method depends on applied shear stress and the used concentration method, supporting results obtained by LO and turbidity measurements (FIG. 3). The increase of immunoglobulin in polymeric form for the concentrated solutions depends on the status of the polymeric precursors before concentration. When material is used, which already contains the immunoglobulin in polymeric form (as in the current example 7.5% as determined by SEC), an increase in the amount of polymeric forms during tangential flow filtration was not detectable by SEC (see Table 2).

TABLE 2
SEC data

| | | | | | | mean | | |
|---|---|---|---|---|---|---|---|---|
| | | | HMW/ | | | value of | | |
| | HMW/ | HMW/ | polymeric | mean | increase | increase | | |
| | polymeric | polymeric | forms | value | in | in | main | |
| | form | form | Peak 1 | polymeric | aggregates | aggregates | Peak | LMW |
| sample | Peak 1 | Peak 2 | and 2 | forms | [%] | [%] | IL-1R | forms |
| before | 0.54 | 0.00 | 0.54 | 0.67 | | | 99.46 | 0.00 |
| concentration, | 0.49 | 0.22 | 0.71 | | | | 98.29 | 0.00 |
| solution | 0.74 | 0.02 | 0.76 | | | | 99.14 | 0.04 |
| containing a | | | | | | | | |
| low number | | | | | | | | |
| of aggregated | | | | | | | | |
| forms | | | | | | | | |
| TFF with Δp = | 0.90 | 0.00 | 0.90 | 0.95 | 34.33 | 41.79 | 99.03 | 0.06 |
| 1.2 bar of | 0.96 | 0.03 | 0.99 | | 47.76 | | 98.93 | 0.00 |
| low number | 0.00 | 0.96 | 0.96 | | 43.28 | | 98.99 | 0.05 |
| aggregate | | | | | | | | |
| solution | | | | | | | | |
| before | 5.07 | 2.20 | 7.27 | 7.23 | | | 92.59 | 0.15 |
| concentration, | 4.69 | 2.50 | 7.19 | | | | 92.64 | 0.18 |
| solution | | | | | | | | |
| containing a | | | | | | | | |
| high number | | | | | | | | |
| of aggregated | | | | | | | | |
| forms | | | | | | | | |
| TFF with Δp = | 5.05 | 2.61 | 7.66 | 7.29 | 5.95 | 0.83 | 92.17 | 0.16 |
| 1.2 bar of | 4.63 | 2.63 | 7.26 | | 0.41 | | 92.56 | 0.18 |
| high number | 4.49 | 2.46 | 6.95 | | −3.87 | | 92.86 | 0.20 |
| aggregate | | | | | | | | |
| solution | | | | | | | | |
| before | 5.27 | 2.41 | 7.68 | 7.44 | | | 92.02 | 0.30 |
| concentration, | 4.69 | 2.50 | 7.19 | | | | 92.64 | 0.18 |
| solution | | | | | | | | |
| containing a | | | | | | | | |
| high number | | | | | | | | |
| of aggregated | | | | | | | | |
| forms | | | | | | | | |
| TFF with Δp = | 5.02 | 2.80 | 7.82 | 8.02 | 5.18 | 7.91 | 91.84 | 0.34 |
| 3.0 bar of | 5.09 | 2.92 | 8.01 | | 7.73 | | 91.81 | 0.19 |
| high number | 5.38 | 2.86 | 8.24 | | 10.83 | | 91.54 | 0.22 |
| aggregate | | | | | | | | |
| solution | | | | | | | | |
| before | 0.59 | 0.15 | 0.74 | 0.73 | | | 99.17 | 0.09 |
| concentration, | 0.49 | 0.22 | 0.71 | | | | 98.29 | 0.00 |
| solution | | | | | | | | |
| containing a | | | | | | | | |
| low number | | | | | | | | |
| of aggregated | | | | | | | | |
| forms | | | | | | | | |
| TFF with Δp = | 0.00 | 1.06 | 1.06 | | 46.21 | 37.47 | 98.83 | 0.11 |
| 3.0 bar of | 0.00 | 0.98 | 0.98 | | 35.17 | | 98.83 | 0.19 |
| low number | 0.00 | 0.95 | 0.95 | | 31.03 | | 99.03 | 0.02 |
| aggregate | | | | | | | | |
| solution | | | | | | | | |

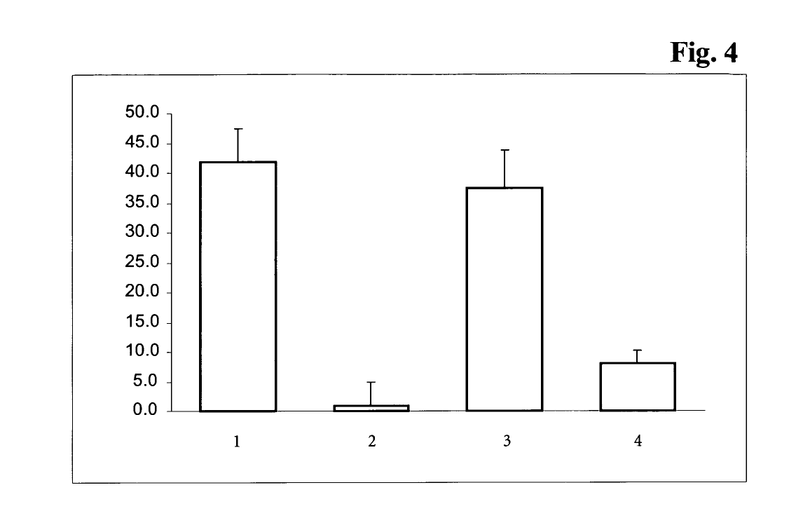

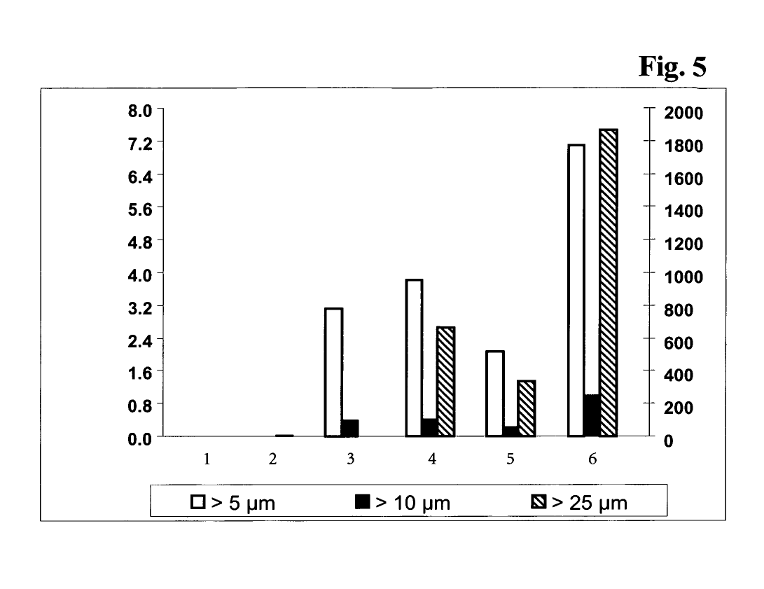

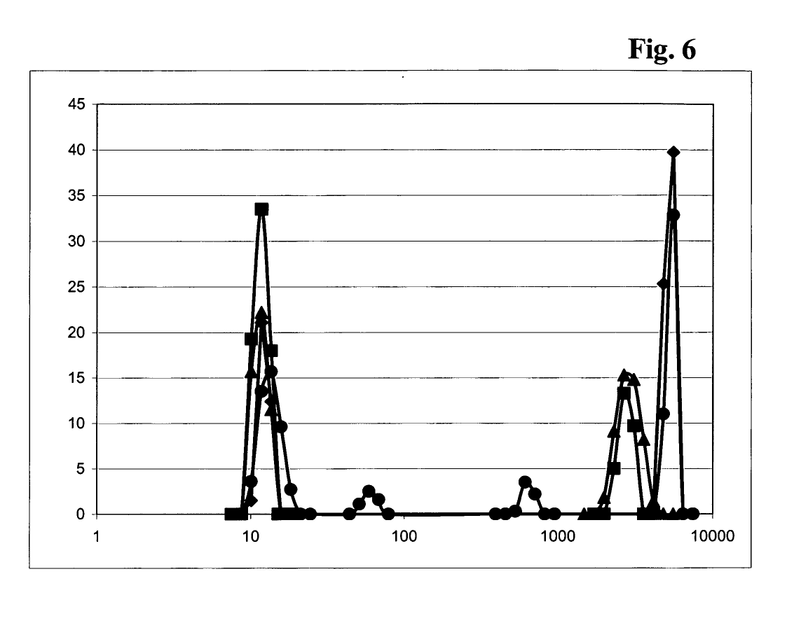

In contrast to this the amount of high molecular weight forms/polymeric soluble forms increased when nearly monomeric material (0.6% of immunoglobulin in polymeric form as determined by SEC) was concentrated (FIG. 4). Regarding the status of larger and insoluble aggregates after concentration a contrarily relation was observed. Solutions with a higher extend of polymeric form precursors before concentration showed more larger and insoluble aggregates compared to the initially nearly monomeric solutions (FIG. 5). A shift from smaller, soluble polymeric precursors, i.e. from polymeric forms in which only a few immunoglobulin molecules are aggregated, to larger insoluble aggregates during the TFF process depending on the status of the polymeric precursors of the used intermediate was found. DLS data supports this perspective (FIG. 6).

Not only unfavorable high pressure profiles in TFF but also contaminants like soluble aggregates can cause an ongoing aggregation process with a shift of aggregates to larger insoluble species and precipitates during concentration by TFF.

1. A method for removing immunoglobulin aggregates from an immunoglobulin solution, comprising:
i) providing a solution containing said immunoglobulin, wherein said immunoglobulin is present in said solution in monomeric and polymeric form, or
providing a solution containing an immunoglobulin, wherein in said solution the formation of said immunoglobulin in polymeric form is induced by the application of heat,
ii) concentrating said solution provided under i) by employing a tangential flow filtration with a constant Δp of 3.0 bar, and a constant transmembrane pressure of 0.6 bar; and
iii) removing immunoglobulin aggregates from the immunoglobulin solution obtained in step ii) by filtration with a 0.2 μm pore size filter,
whereby the number of particles in said concentrated solution of step ii) with a size of more than 1 μm is smaller than 200 times the number of particles with a size of more than 1 μm in the solution prior to the concentrating when determining the number of particles by light obscuration.

2. The method of claim 1, wherein the number of particles in said concentrated solution of with a size of more than 5 μm is smaller than 100 times the number of particles with a size of more than 5 μm in the solution prior to the concentrating when determining the number of particles by light obscuration.

3. The method of claim 1, wherein the fraction of said polymeric soluble immunoglobulin form is more than 5% when determined as the area under the curve of the peaks eluted prior to the peak of the monomeric immunoglobulin in a size exclusion chromatogram of said solution.

4. The method of claim 1, wherein the heterologous immunoglobulin is a complete immunoglobulin, or an immunoglobulin fragment, or an immunoglobulin conjugate.

5. The method of claim 1, wherein the determining by light obscuration is at a protein concentration of 90 mg/ml.

6. The method of claim 2, wherein the determining by light obscuration is at a protein concentration of 90 mg/ml.

7. The method of claim 3, wherein the determining by light obscuration is at a protein concentration of 90 mg/ml.

8. The method of claim 4, wherein the determining by light obscuration is at a protein concentration of 90 mg/ml.
